(19) United States
(12) Patent Application Publication
(10) Pub. No.: US 2009/0270494 A1
(43) Pub. Date: Oct. 29, 2009

(54) Salts of Dihydroxyanthraquinone Carboxylic Acids and Their Therapeutic Use

(76) Inventor: Andrea Walmsley, Bedfordshire (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/513,819

(22) PCT Filed: Nov. 9, 2007
(86) PCT No.: PCT/GB2007/004280

§ 371 Date: Jun. 16, 2009

(30) Foreign Application Priority Data
Nov. 10, 2006 (GB) 0622479.4

(51) Int. Cl. A61K 31/351; C07D 309/32

(52) U.S. Cl. 514/451; 549/427

(57) ABSTRACT

A compound of general formula (I): wherein Xi is H or COR₁ and X₂ is H or COR₂ but X₁ and X₂ are not both H; R₁ and R₂ are the same or different and are each C₁₋₄ alkyl substituted with R₃, or a four to seven-membered ring which can be optionally substituted with R₈ and can contain one or more additional heteroatoms selected from O, S(O)n and NR₉; R₃ is F, CF₃, OR₄, NR₅R₆ or S(O)ₙR₇; R₄, R₅ and R₆ are the same or different and are each H or C₁₋₄ alkyl optionally substituted with R₃, or NR₅R₆ is a C₄₋₆ heterocycloalkyl ring containing one or more heteroatoms selected from O, NR₈ and S(O)ₙ; each n is 0-2; R₇ is C₁₋₄ alkyl; R₈ is as defined for R₃ or C₁₋₄ alkyl optionally substituted with R₃ or halogen; R₉ is H or C₁₋₄ alkyl; Y is NR₉R₁₀R₁₁; and R₁₀ and R₁₁, are the same or different and are each H or C₁₋₆ alkyl optionally substituted with R₃ or halogen, or NR₁₀R₁₁, is a four to seven membered ring which can be optionally substituted with R₈ or COR₁, and can contain one or more additional heteroatoms selected from O, S(O)ₙ and NR₉; and hydrates thereof.

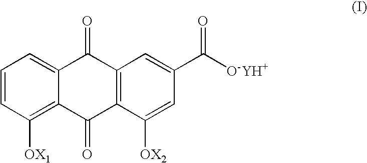

### FIELD OF THE INVENTION

The present invention relates to novel salts of dihydroxyanthraquinone carboxylic acid derivatives which are ester derivatives of rhein, and to their therapeutic use.

### BACKGROUND OF THE INVENTION

WO2005/085170 describes ester derivatives of rhein and their use in a wide range of anti-inflammatory conditions, including rheumatoid arthritis, osteoarthritis, osteoporosis, Crohn's disease, ulcerative colitis, multiple sclerosis, periodontitis, gingivitis, graft versus host reactions, psoriasis, scleroderma, atopic dermatitis, asthma, systemic lupus erythematosus (SLE), nephropathy and chronic obstructive pulmonary disease (COPD).

### SUMMARY OF THE INVENTION

The present invention is related to the observation that salt derivatives of formula (I) exhibit improved physical and chemical parameters such as solubility and stability over the free base. These salts have clinical utility in the wide range of inflammatory and autoimmune diseases.

In a first aspect of the invention, novel compounds are of general formula (I):

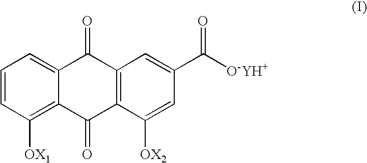

wherein X₁ is H or COR₁ and X₂ is H or COR₂ but X₁ and X₂ are not both H;

R₁ and R₂ are the same or different and are each C₁₋₄ alkyl substituted with R₃, or a four to seven-membered ring which can be optionally substituted with R₈ and can contain one or more additional heteroatoms selected from O, S(O)ₙ and NR₉;

R₃ is F, CF₃, OR₄, NR₅R₆ or S(O)ₙR₇;

R₄, R₅ and R₆ are the same or different and are each H or C₁₋₄ alkyl optionally substituted with R₃, or NR₅R₆ is a C₄₋₆ heterocycloalkyl ring containing one or more heteroatoms selected from O, NR₈ and S(O)ₙ;

each n is 0-2;

R₇ is C₁₋₄ alkyl;

R₈ is as defined for R₃ or C₁₋₄ alkyl optionally substituted with R₃ or halogen;

R₉ is H or C₁₋₄ alkyl;

Y is NR₉R₁₀R₁₁; and

R₁₀ and R₁₁ are the same or different and are each H or C₁₋₆ alkyl optionally substituted with R₃ or halogen, or may form part of a four to seven membered ring which can be optionally substituted with R₈ or COR₁, and can contain one or more additional heteroatoms selected from O, S(O)ₙ and NR₉;

and hydrates thereof.

Compounds of the invention may be diesters (X is COR₁ and X₂ is COR₂; hereinafter formula 1) or monoesters where X₁ is H (formula 2) or X₂ is H (formula 3).

Preferred compounds are those where Y=arginine, histidine, lysine, diethanolamine, diethylamine, diethylaminoethanol, dimethylaminomethanol, ethanolamine, ethylenediamine, imidazole, 4-(2-hydroxyethyl)-morpholine, N-methyl-glucamine, piperazine, 1-(2-hydroxyethyl)-pyrrolidine, triethanolamine or tromethamine. Most preferred compounds are those where Y=tromethamine or 4-(2-hydroxyethyl)-morpholine.

Further aspects of the invention include pharmaceutical compositions comprising the compounds of formula I, their use in therapy and, more particularly, their use in the treatment of inflammatory conditions.

Carboxylic acid salts of formula (I) inhibit cytokine production and T-cell proliferation, and are therefore of utility in the treatment of T-cell mediated diseases including those described above.

### DESCRIPTION OF THE INVENTION

It will be appreciated that the compounds according to the invention can contain one or more asymmetrically substituted carbon atoms. The presence of one or more of these asymmetric centres in a compound of formula (1), can give rise to stereoisomers, and in each case the invention is to be understood to extend to all such stereoisomers, including enantiomers and diastereomers, and mixtures including racemic mixtures thereof.

The term “C₁₋₄ alkyl” refers to a straight or branched chain alkyl moiety having from one to four carbon atoms, including for example, methyl, ethyl, propyl, isopropyl, butyl, tert-butyl and the like.

The term “C₁₋₆ alkyl” refers to a straight or branched chain alkyl moiety having from one to four carbon atoms, including for example, methyl, ethyl, propyl, isopropyl, butyl, tert-butyl and the like.

The term “C₄₋₆ heterocycloalkyl” refers to a saturated heterocyclic moiety having from three to six carbon atoms and one or more heteroatom from the group N, O, S and includes for example azetidinyl, oxetidinyl, pyrrolidinyl, tetrahydrofuranyl, piperidinyl, tetrahydropyranyl and the like.

The term “halogen” means fluorine, chlorine, bromine or iodine.

The preparation of the free base compounds of general formula (1), including experimental detail, is described in WO2005/085170. The preparation of salts of compounds of formula (1) can be achieved by performing a range of experiments under various crystallising conditions, such as evaporative crystallisation, temperature variation and slurry ripening, and which will be familiar to those skilled in the art. A selection of salt formers has been used in experiments, including 4-(2-hydroxyethyl)morpholine and tromethamine, which were characterised in more detail by a range of techniques including XRPD and ¹H NMR.

For the treatment of rheumatoid arthritis, multiple sclerosis, and in other diseases and indications resulting from the over-activity of T-cells such as those highlighted above, the compounds of formula (1) may be administered orally, topically, parenterally, by inhalation or nasal spray or rectally in dosage unit formulations containing non-toxic pharmaceutically acceptable carriers, adjuvants and vehicles. The term parenteral as used herein includes subcutaneous injections, intravenous, intramuscular, intrasternal injection or infusion techniques. In addition to the treatment of warm-blooded animals such as mice, rats, horses, cattle, sheep, dogs, cats etc, the compounds of the invention are effective in the treatment of humans.

A pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. No. 4,256,108, U.S. Pat. No. 4,166,452 and U.S. Pat. No. 4,265,874 to form osmotic therapeutic tablets for control release.

Formulations for oral use may also be presented as hard gelatin capsules where in the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate polyvinyl-pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl or n-propyl, p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified, for example sweetening, flavouring and colouring agents, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be in a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of formulae (1) may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc containing the compounds of formulae (1) are employed. For purposes of this specification, topical application includes mouth washes and gargles.

Dosage levels of the order of from about 0.05 mg to about 140 mg per kilogram of body weight per day are useful in the treatment of the above-indicated conditions (about 2.5 mg to about 7 g per patient per day). For example, inflammation may be effectively treated by the administration of from about 0.01 to 50 mg of the compound per kilogram of body weight per day (about 0.5 mg to about 3.5 g per patient per day).

The amount of active ingredient that may be combined with the carrier materials to produce a single dosage form will vary depending upon the host treated and the particular mode of administration. For example, a formulation intended for the oral administration of humans may vary from about 5 to about 95 percent of the total composition. Dosage unit forms will generally contain between from about 1 mg to about 500 mg of an active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

The following Examples illustrate the invention. All starting materials were subjected to extended drying prior to use.

### Example 1

### 4-(2-Hydroxyethyl)morpholine salt of 4a,9,9a,10-tetrahydro-4,5-bis(2-[tetrahydro-2H-pyran-4-yl]-2-oxoethyl)-9,10-dioxoanthracene-2-arboxylic acid

4a,9,9a,10-Tetrahydro-4,5-bis(2-[tetrahydro-2H-pyran-4-yl]-2-oxoethyl)-9,10-dioxoanthracene-2-carboxylic acid (111.6 mg) and 4-(2-hydroxyethyl)morpholine (“the salt former”, 26.9 μl) were stirred for 1 day in THF (30 ml), temperature cycle (max/min: 60° C./10° C. (both kept >2 hours)), concentrated under dry N₂ flow to a suspension volume of 1-2 ml before being filtered and dried under vacuum at room temperature.

¹H NMR (DMSO): 1.7-1.9 (4H, m), 2.0 (4H, m), 2.5 (DMSO NMR solvent), 2.6-2.7 (6H, m morpholine ethyl group: CH₂—N), 3.0 (2H, m), 3.5 ([4]¹H, t morpholine group: CH₂O), 3.6 ([2]¹H, ethyl group: CH₂O), 3.7 ([4]¹H, m), 3.8 (H₂O residual solvent), 4.0 ([4]¹H, m), 7.6 (1H, d), 7.9 (1H, t), 8.0 (1H, s), 8.1 (1H, d), 8.6 (1H, s).

This crystalline form exhibits a characteristic X-ray powder diffraction pattern with characteristic peaks expressed in d-values (Angstrom): 27.7 (vw); 23.5 (s); 11.6 (m); 10.4 (m); 9.7 (w); 8.8 (vw); 7.7 (vw); 5.62 (w); 5.44 (w); 5.30 (w); 5.17 (s); 5.03 (vw); 4.83 (w); 4.63 (s); 4.53 (m); 4.38 (w); 4.25 (m); 4.02 (s); 3.91 (s); 3.77 (w); 3.72 (w); 3.63 (vw); 3.55 (w); 3.38 (w); 3.31 (vw); 3.23 (w); 3.15 (w); 3.05 (w).

### Example 2

### Tromethamine salt of 4a,9,9a,10-tetrahydro-4,5-bis(2-[tetrahydro-2H-pyran-4-yl]-2-oxoethyl)-9,10-dioxoanthracene-2-carboxylic acid

4a,9,9a,10-Tetrahydro-4,5-bis(2-[tetrahydro-2H-pyran-4-yl]-2-oxoethyl)-9,10-dioxoanthracene-2-carboxylic acid (112.1 mg) and 26.7 mg of tromethamine (“the salt former”) were stirred in THF (30 ml) for 1 day, temperature cycle (max/min: 60° C./10° C. (both kept >2 hours)), concentrated under dry N₂ flow to a suspension volume of 1-2 ml before being filtered and dried under vacuum at room temperature.

¹H NMR (DMSO): 1.7-1.9 (4H, m), 2.0 (4H, m), 2.5 (DMSO NMR solvent), 3.0 (2H, m), 3.4 (water, residual solvent), 3.4 ([4H]² m), 3.9 (4H, m), 7.6 (1H, d), 7.9 (1H, s), 7.9 (1H, t), 8.1 (1H, d), 8.5 (1H, s).

This crystalline form exhibits a characteristic X-ray powder diffraction pattern with characteristic peaks expressed in d-values (Angstrom): 21.3 (m); 11.3 (s); 10.6 (vw); 9.0 (vw); 8.2 (vw); 7.1 (vw); 6.9 (m); 5.78 (vw); 5.65 (vw); 5.48 (vw); 5.37 (w); 5.30 (vw); 5.11 (m); 4.90 (vw); 4.79 (vw); 4.59 (vw); 4.52 (w); 4.37 (m); 4.24 (m); 4.15 (m); 3.88 (w); 3.75 (vw); 3.66 (w); 3.50 (vw); 3.40 (vw); 3.29 (vw); 3.18 (vw); 3.15 (vw); 3.02 (vw).

1. A compound of general formula (I): 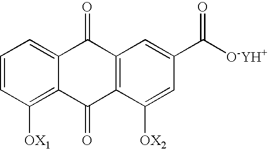
wherein X₁ is H or COR₁ and X₂ is H or COR₂ but X₁ and X₂ are not both H;
R₁ and R₂ are the same or different and are each C₁₋₄ alkyl substituted with R₃, or a four to seven-membered ring which can be optionally substituted with R₈ and can contain one or more additional heteroatoms selected from O, S(O)ₙ and NR₉;
R₃ is F, CF₃, OR₄, NR₅R₆ or S(O)ₙR₇;
R₄, R₅ and R₆ are the same or different and are each H or C₁₋₄ alkyl optionally substituted with R₃, or NR₅R₆ is a C₄₋₆ heterocycloalkyl ring containing one or more heteroatoms selected from O, NR₈ and S(O)ₙ;
each n is 0-2;
R₇ is C₁₋₄ alkyl;
R₈ is as defined for R₃ or C₁₋₄ alkyl optionally substituted with R₃ or halogen;
R₉ is H or C₁₋₄ alkyl;
Y is NR₉R₁₀R₁₁; and
R₁₀ and R₁₁ are the same or different and are each H or C₁₋₆ alkyl optionally substituted with R₃ or halogen, or NR₁₀R₁₁ is a four to seven membered ring which can be optionally substituted with R₈ or COR₁, and can contain one or more additional heteroatoms selected from O, S(O)ₙ and NR₉;
and hydrates thereof.

2. The compound of claim 1, wherein X₁ is COR₁ and X₂ is COR₂.

3. The compound of claim 1, wherein X₁ is H and X₂ is COR₂.

4. The compound of claim 1, wherein X₁ is COR₁ and X₂ is H.

5. The compound of claim 1, wherein R₃ is CF₃, OR₄, NR₅R₆ or S(O)ₙR₇.

6. The compound of claim 1, which is a 4-(2-hydroxyethyl)morpholine
salt.

7. The compound of claim 1, which is a tromethamine salt.

8. The compound of claim 1, which is a salt of 4,5-ditetrahydropyranoyloxy-9,10-dioxo-9,10-dihydroanthracene-2-carboxylic acid.

9. The compound of claim 8, which is the 4-(2-hydroxyethyl)-morpholine salt.

10. The compound of claim 9, which is the tromethamine salt.

11. A pharmaceutical composition, comprising a compound of claim 1 and a pharmaceutically acceptable diluent or carrier.

12. A method for the treatment or prevention of a condition associated with T-cell proliferation or that is mediated by pro-inflammatory cytokines wherein said method comprises administering to a subject in need of such treatment or prevention, a compound according to claim 1.

13. The method according to claim 12, wherein the condition is a chronic degenerative disease.

14. The method according to claim 12, wherein the condition is a chronic demyelinating disease.

15. The method according to claim 12, wherein the condition is a respiratory disease.

16. The method according to claim 12, wherein the condition is an inflammatory bowel disease (IBD).

17. The method according to claim 12, wherein the condition is a dermatological condition.

18. The method according to claim 12, wherein the condition is periodontal disease or gingivitis.

19. The method according to claim 12, wherein the condition is diabetic nephropathy, lupus nephritis, IgA nephropathy or glomerulonephritis.

20. The method according to claim 12, wherein the condition is systemic lupus erythematosus (SLE).

21. The method according to claim 12, wherein the condition is graft vs host disease.
